(19) United States
(12) Patent Application Publication
(10) Pub. No.: US 2021/0153778 A1
(43) Pub. Date: May 27, 2021

(54) SMART APPAREL FOR MONITORING ATHLETICS AND ASSOCIATED SYSTEMS AND METHODS

(71) Applicant: Intel Corporation, Santa Clara, CA (US)

(72) Inventors: Anupama Gupta, Santa Clara, CA (US); Timothy Hansen, Folsom, CA (US); Lili Jiang, Beijing (CN); Todd Johnson, Santa Clara, CA (US); Gary Kwan, San Jose, CA (US); Wenlong Li, Beijing (CN); Yu-Wei Liao, Santa Clara, CA (US); Bhaveshkumar Makwana, Sunnyvale, CA (US); Alok Mishra, Cupertino, CA (US); Kisang Pak, Winchester, VA (US); Mary Smiley, Santa Clara, CA (US); Sun Hee Wee, Santa Clara, CA (US); Johnny Yip, Sunnyvale, CA (US)

(21) Appl. No.: 16/630,352

(22) PCT Filed: Aug. 10, 2017
(86) PCT No.: PCT/CN2017/096772

§ 371 Date: Jan. 10, 2020

(51) Int. Cl. A61B 5/11; A61B 5/00

(52) U.S. Cl. CPC A61B 5/1114; A61B 5/0022; A61B 5/1123; A61B 5/1126; A61B 2562/0219; A61B 5/7435; A61B 2503/10; A61B 2505/09; A61B 5/6802

(57) ABSTRACT

Smart apparel for monitoring athletics and associated systems and methods are disclosed. An example apparatus includes a data interface to access first motion data and second motion data generated by the smart apparel, the first motion data associated with a first joint on a body and the second motion data associated with a second joint on the body; a motion data fuser to fuse the first motion data and the second motion data; an analytics determiner to process the fused first and second motion data to identify a progression of a motion based activity; and a display organizer to generate a graphical display representing the progression of the motion based activity.

### FIELD OF THE DISCLOSURE

This disclosure relates generally to smart apparel, and, more particularly, to smart apparel for monitoring athletics and associated systems and methods.

### BACKGROUND

There are many type(s) of athletics including sports, dance, fitness, training, etc. Some sports are swing-based. Example swing-based sports include, but are not limited to, golf, baseball and tennis. In golf, a player attempts to strike a ball with a club. In baseball, a batter attempts to hit a ball with a bat. In tennis, a player attempts to strike a ball with a racket. Other athletic events involve other swinging motions. For example, cross-fit often involves swinging kettlebells.

### BRIEF DESCRIPTION OF THE DRAWINGS

FIG. 1 is a schematic illustration of an example system constructed in accordance with the teachings of this disclosure to obtain and process data associated with athletics to generate results associated with the same.

FIG. 2 is a schematic illustration of an example implementation of the motion monitor of FIG. 1.

FIG. 3 is a schematic illustration of an example implementation of the motion data analyzer of FIG. 1.

FIG. 4 illustrates an example implementation of the example smart apparel of FIG. 1.

FIGS. 5-13 illustrate example results that can be displayed by the mobile device of FIG. 1.

FIG. 14 illustrates a first example image and/or video that can be obtained and/or displayed by the example mobile device of FIG. 1.

FIG. 15 illustrates a second example image and/or video displayed by the example mobile device of FIG. 1 and annotated with example results including performance indicators and metrics by the example system of FIG. 1.

FIG. 16 illustrates a third example image and/or video displayed by the example mobile device of FIG. 1 and annotated with example results including performance indicators and metrics by the example system of FIG. 1.

FIGS. 17 and 18 are flowcharts representative of example machine readable instructions that may be executed to implement the motion monitor of FIGS. 1 and/or 2.

FIGS. 19 and 20 are flowcharts representative of example machine readable instructions that may be executed to implement the motion data analyzer of FIGS. 1 and/or 3.

FIG. 21 is a processor platform structured to execute the instructions of FIGS. 17 and 18 to implement the motion monitor of FIGS. 1 and/or 2.

FIG. 22 is a processor platform structured to execute the instructions of FIGS. 19 and 20 to implement the motion data analyzer of FIGS. 1 and/or 3.

The figures are not to scale. Wherever possible, the same reference numbers will be used throughout the drawing(s) and accompanying written description to refer to the same or like parts.

### DETAILED DESCRIPTION

Examples disclosed herein relate to smart apparel for monitoring athletic performance. Example smart apparel disclosed herein capture body kinetics (e.g., whole body kinetics) for athletic(s) and/or the like based on bio-mechanic movement points in the body. Such example smart apparel may be used to monitor and/or diagnose movement based activities, such as, for example, action(s) associated with athletics, such as sports. For example, the smart apparel may be used to capture body kinetics related to throwing a baseball, hitting a baseball, hitting a softball, throwing a football, etc. However, examples disclosed herein can be used in connection with any movement-based activity. For instance, examples disclosed herein can be used to monitor and/or diagnose movements in dance, such as ballet.

The smart apparel may be washable and/or wearable as day-to-day clothing without modifying any equipment used in association with the apparel. In some disclosed examples, the smart apparel is implemented with sensors positioned at appropriate locations and/or causal data points that monitor the motion of a swing, body mechanics, kinematics, batting mechanics, linear movement, rotational movement, etc. The sensors may be housed within the apparel.

For example, to provide tracking of movement(s) of the wrist, shoulder and hip when the smart apparel is implemented as a smart apparel, the smart apparel constructed in accordance with the teachings of this disclosure includes an example hip sensor disposed at the left hip, an example shoulder sensor disposed at the left shoulder, and an example wrist sensor disposed at the left wrist. While in this example the sensors are disposed on the left side of the smart apparel, the sensors may additionally or alternatively be on the right hip, the right wrist and/or the right shoulder of the example smart apparel. Such an approach provides a complete data set of the torso, hip and arm movement. In the context of monitoring baseball players, using sensors on both sides enables monitoring of both right-handed players and left-handed players, and ambidextrous players (e.g., switch hitters). However, in some examples, sensors disposed on one side of the smart apparel may obtain data from both right-handed players and left-handed players.

The hip sensor, the shoulder sensor and/or the wrist sensor may be coupled (e.g., directly coupled, indirectly coupled, wirelessly coupled) to communicate using an inter-integrated circuit (I²C) protocol and/or any other protocol. In some examples, the hip sensor, the shoulder sensor and/or the wrist sensor are directly coupled using a thermoplastic (TPE)-based wrapper that deters ingress of fluid and/or debris (e.g., sweat ingress, water ingress, etc.) into the wrapper. The sensors may additionally or alternatively be encased in and/or include TPE to deter ingress of debris and/or fluid into the sensors.

In the illustrated examples, the TPE-based wrapper is coupled (e.g., stitched) to the clothing. In some such examples, the TPE-based wrapper may be stitched on the apparel from the left hand, to the left shoulder and to the left hip. In some examples, a battery is included in the TPE wrapper. The battery may be proximate to at least one of the example hip sensor, the example shoulder sensor and the example wrist sensor to provide power to the sensors. In some examples in which the hip sensor is disposed in a housing, a battery may be housed within the housing proximate the hip sensor.

In some examples, the hip sensor is implemented by an accelerometer and/or a gyroscope (e.g., a low power, low noise, 6-axis, inertial measurement unit) to enable the hip sensor to obtain motion data (e.g., movement data) reflecting motion of the hip. The motion data collected by the hip may include, but is not limited to, acceleration data reflecting acceleration of the hip, rotation data reflecting rotation of the hip and/or position data (e.g., spatial position data) reflecting horizontal and/or vertical translation of the hip. In some examples, the shoulder sensor is implemented by an accelerometer and/or a gyroscope (e.g., a low power, low noise, 6-axis, inertial measurement unit) to enable the shoulder sensor to obtain motion data reflecting rotation of the shoulder. The motion data collected by the shoulder sensor may include, but is not limited to, acceleration data reflecting acceleration of the shoulder, rotation data reflecting rotation of the shoulder and/or position data (e.g., spatial position data) reflecting horizontal and/or vertical translation of the shoulder. In some examples, the wrist sensor includes, but is not limited to, an accelerometer and/or a gyroscope (e.g., a 6-axis motion tracking sensor) to enable the wrist sensor to obtain motion data reflecting movement of the wrist including acceleration data reflecting acceleration of the wrist, rotation data reflecting rotation of the wrist and/or position data (e.g., spatial position data) reflecting the position of the wrist.

As noted above, example smart apparel disclosed herein is instructed to collect many types of motion data to provide a complete picture of wrist, hip and shoulder movement. However, in some examples, it may be desirable to focus on a subset of the motion data. For instance, when seeking to monitor and/or improve a swing motion in a swing-based sport, it may be useful to filter non-swing related data from the collected motion data. The non-swing data may be filtered from the motion data by comparing the motion data to reference motion data and removing any data not associated with a reference motion (e.g., a particular swing). In some examples, the non-swing motion data includes movement reflecting movement of the wrists but does not include motion data reflecting movement of the shoulder and/or hips. Alternatively, the non-swing motion data includes movement reflecting movement of the hips but does not include motion data reflecting movement of the shoulder and/or wrists.

To identify swing data and/or non-swing data within the motion data collected by the sensors, wrist acceleration may be compared to reference wrist acceleration associated with a particular movement to be monitored (e.g., a swing) to determine if the wrist acceleration satisfies a threshold of the reference wrist acceleration (e.g., the wrist acceleration is greater than a particular amount). When the wrist acceleration satisfies the threshold, in some examples, a swing is identified as taking place. When the wrist acceleration does not satisfy the threshold, in some examples, it is determined that a swing is not taking place. While monitoring wrist acceleration is mentioned as one example of how to determine when a swing is taking place and when a swing is not taking place, other examples exist. For example, acceleration and/or rotation data reflecting acceleration and/or rotation of one or more of the wrist, the shoulder and/or the hip may be compared to reference data to determine if the monitored acceleration and/or rotation data satisfies a threshold indicating that a swing taking place. Once identified, in some examples, the non-swing data may be removed, filtered and/or parsed from the motion data.

To enable the motion data to be accessed by a mobile device and/or a computer (e.g., a virtual machine and/or service in the cloud, a computer at a remote facility, etc.) for further processing, in some examples, the smart apparel includes a transceiver or the like. For example, the smart apparel may be provided with communication circuitry and supporting software/firmware to transmit and/or receive commands and/or data via any past, present or future communication protocol (e.g., cellular; Wi-Fi; and/or Bluetooth).

In some examples, the orientation of the sensors throughout a motion (e.g., a swing) is determined by fusing acceleration data with rotation data and/or position data collected by the sensor(s). The data may be fused using an inertial measurement unit algorithm and/or another fusion algorithm. In some examples, analytics are performed on the fused data and/or the individual motion data to identify posture-specific metrics, key performance indicators and/or other metrics associated with the motion (e.g., the swing). The posture-specific metrics, the key performance indicators and/or the other metrics may be specific to, and/or associated with, any movement and/or activity being monitored.

The key performance indicators may include bio-kinetic feedback (e.g., full-body bio-kinetic feedback) and/or bio-kinetic performance indicators that focus on causes and/or coordinated movement of portions of the body relevant to the action being performed (e.g., throwing a football, hitting a baseball, etc.). In some examples, one or more key performance indicators are determined by characterizing a progression of a movement (e.g., a swing) based on an angular velocity profile. In some examples, one or more key performance indicators are based on a degree of correspondence (e.g., alignment in time) between velocity peaks detected by the different sensors. For example, the progression of a swing may be analyzed by comparing and/or combining motion data from the hip sensor and one or more of the shoulder sensor and/or the wrist sensor to determine how the hip is moving in relation to the shoulder. This relationship may be considered spatially (e.g., positional differences), temporarily (e.g., times at which peaks occur) and/or both spatially and temporally (e.g., comparison of rates of positional changes). Thus, the progression of the swing may be analyzed by combining motion data from the hip sensor and one or more of the shoulder sensor and/or the wrist sensor to determine the position of the hip relative to the shoulder at each phase of the swing.

While the key performance indicators may include any type of indicator(s), in some examples, the key performance indicators include hip speed, hip rotation, shoulder speed, shoulder rotation, hand speed, hand rotation, forward lean, lateral tilt, hand path side view, hand path top view, torso flexion and/or maximum separation. In some examples, the key performance indicators are based on a chain of movements (e.g., a combination of relative actions such as hip speed, shoulder dip and hand rotation) leading to a result (e.g., hitting a ball). Thus, examples disclosed herein provide contextual feedback for athletic movements in an athletic endeavor such as swing-based sports and/or throw-based sports to enable participants to improve and/or change their movement(s) (e.g., how they hit and/or throw a ball) to improve performance in the movement-based activity being monitored. Focusing on the movements leading up to the result of the movements may provide a detailed view into factors that negatively or possibly affect the result. Such detailed information may assist in making adjustments to specific components of the motion to significantly improve the overall result. For example, analyzing detailed motion data (e.g., hip movement, shoulder movement and/or wrist movement) instead of the result (e.g., resultant bat speed), enables the focus to be on the many factors that cause the result (e.g., a suboptimal swing) instead of the resulting effect (e.g., bat speed). This enables adjustments in a much more specific manner (e.g., turn your hips earlier) than a general observation (e.g., you swing behind the ball). As such, examples disclosed herein provide detailed feedback on the causal actions leading to a result in an athletic motion. This detailed feedback may enable focus on specific components of a motion that can lead to improved results for the overall motion.

In some examples, image and/or video data is obtained and associated with key performance indicators and/or metrics identified by the system. To enable past movements and/or performances to be compared, in some examples, the image and/or video data and associated results (e.g., the key performance indicators, metrics, etc.) are compared to historical data to enable side-by-side motion comparisons. In some examples, the key performance indicators and/or other metrics are shown overlaying and/or annotating the image and/or video data. In some examples, telestration (e.g., annotation with a finger or writing instrument) is performable on the image and/or video data.

While the above examples mention swinging a baseball bat as an example of a swing-based sport, the examples disclosed herein can be implemented in any other athletic action, such as, for example, football, golf, tennis, bowling, swimming, baseball throwing/pitching, skiing, dancing, skating, etc.

Example smart apparel disclosed herein is usable to capture whole body kinetics including the coordinated muscle movements for the entire body. Thus, although the above describes the smart apparel as a smart apparel, the smart apparel may be implemented as pants, shorts, gloves, etc. For instance, in monitoring the throwing of a baseball, example smart apparel disclosed herein capture the entire motion progression from lifting a lead foot through the progression of movement in the hips, the trunk and the upper body including the flexion of the knees and/or elbows. In some such examples, the smart apparel may include differently placed sensors to capture motion data. For example, to capture body kinematics (e.g., full body kinematics) for throwing a baseball, the smart apparel may include a foot sensor carried by a shoe or sock, a knee sensor carried by pants or shorts and/or an elbow sensor on the sleeve of the jacket or shirt. Of course, different sensors may be used that are placed in different locations for different body parts when participating in the movement based activities being monitored.

In some examples, a foot sensor obtains motion data relating to and/or reflecting movement of a foot (e.g., data representing acceleration of the foot, rotation of the foot and/or spatial position of the foot). In some examples, a knee sensor obtains motion data relating to and/or reflecting movement of the knee (e.g., data representing acceleration of the knee, rotation of the knee and/or spatial position of the knee over time). In some examples, an elbow sensor obtains motion data relating to and/or reflecting movement of the elbow (e.g., data representing acceleration of the elbow, rotation of the elbow and/or spatial position of the elbow over time). While the above example mentions the smart apparel including a foot sensor, a knee sensor and an elbow sensor, sensors to obtain motion data may be placed in any location on the body depending on the movement based activities being monitored.

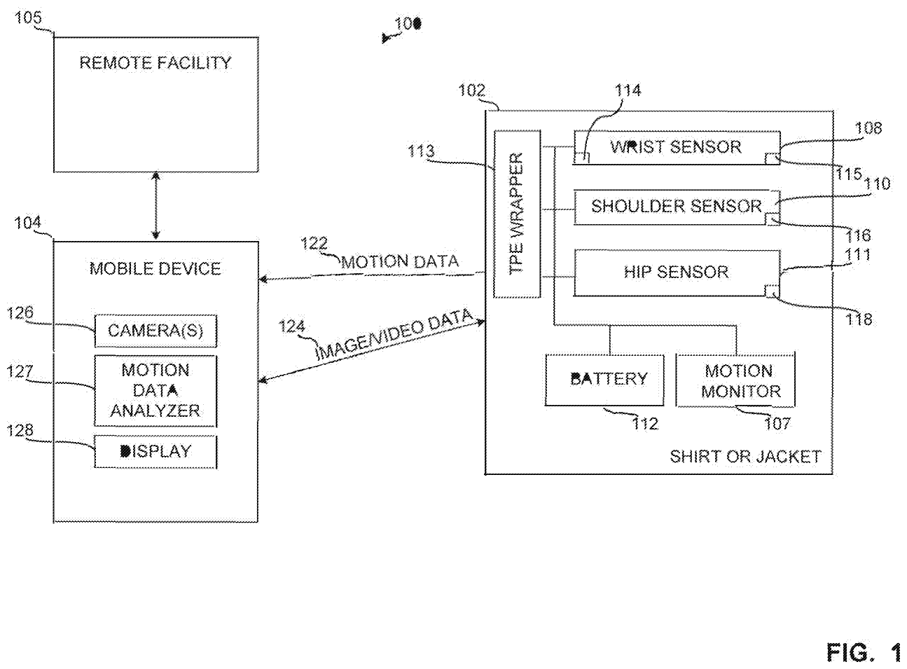

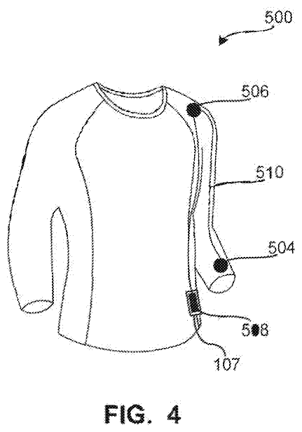

FIG. 1 is a schematic illustration of an example smart apparel system 100 implemented in accordance with the teachings of this disclosure capture body kinetics and/or action(s) based on bio-mechanic movement points on the body. In this example, the system 100 includes example smart apparel 102, an example mobile device 104 and an example remote facility 105. While the smart apparel 102 included in the example system 100 of FIG. 1 is illustrated as a type of smart apparel in which the examples disclosed herein can be implemented, in other examples, the system 100 includes additional and/or alternative pieces of apparel (e.g., pants, socks, shorts, headwear and/or footwear). For example, the example system 100 may additionally and/or alternatively include shoe(s), boot(s) (e.g., example ski boots), shorts, pants, glove(s) and/or headwear (e.g. a helmet, a hat, etc.) or, more generally, any type of apparel. FIG. 4 illustrates an example smart apparel (e.g., a pull over) implementation of the smart apparel 102.

While the example system 100 may be used to monitor any type of movement based activity, in the following, the example system 100 is described in the context of capturing kinetics associated with hitting a baseball. In such examples, the smart apparel 102 includes an example motion monitor 107, an example wrist sensor 108, an example shoulder sensor 110, an example hip sensor 111 and an example battery 112. To couple the wrist sensor 108, the shoulder sensor 110 and the hip sensor 111 and/or to enable communication therebetween, in this example, the smart apparel 102 includes an example TPE wrapper 113. The motion monitor 107, the wrist sensor 108, the shoulder sensor 110 and/or the hip sensor 111 may be housed within the smart apparel 102. Alternatively, the motion monitor 107 may be remote to the smart apparel 102.

To enable motion data reflecting movement of the wrist to be obtained when the smart apparel 102 is being worn by an individual and/or athlete, the smart apparel 102 includes a housing containing an example motion sensor 114. In some examples, the motion sensor 114 is implemented by one or more of an accelerometer, a gyroscope and/or a 6-axis motion tracking sensor to collect motion data representative of the wrist such as acceleration data, rotation data and/or spatial position data. In the illustrated example, to enable a status (e.g., powered on) of the wrist sensor 108 to be displayed, the example wrist sensor 108 includes an example display 115 that may be implemented as a light, a light emitting diode (LED), etc.

To enable motion data reflecting movement of the shoulder to be obtained when the smart apparel 102 is being worn by an individual and/or athlete, in some examples, the shoulder sensor 110 includes an example motion sensor 116 contained in a housing. In some examples, the motion sensor 116 is implemented by one or more of an accelerometer, a gyroscope and/or a low power, low noise, 6-axis, inertial measurement unit to collect motion data representative of motion of the shoulder such as acceleration data, rotation data and/or spatial position data.

To enable motion data reflecting movement of the hip to be obtained when the smart apparel 102 is being worn by an individual and/or athlete, in some examples, the hip sensor 111 includes an example motion sensor 118 contained in a housing. In some examples, the motion sensor 118 is implemented by one or more of an accelerometer, a gyroscope and/or a low power, low noise, 6-axis, inertial measurement unit to collect motion data representative of motion of the hip such as acceleration data, rotation data and/or spatial position data.

In operation, an individual and/or athlete may wear the smart apparel 102 when taking a swing at a baseball. During and/or throughout the swing, the wrist sensor 108 captures the acceleration of the wrist, rotation of the wrist and/or position of the wrist. In some examples, the acceleration data is representative of acceleration of the wrist, the rotation data is representative of rotation of the wrist and/or the position data is representative of the position of the wrist is provided to the motion monitor 107. Additionally or alternatively, during and/or throughout the swing, in some examples, the shoulder sensor 110 captures the acceleration of the shoulder, rotation of the shoulder and/or position of the shoulder. In some examples, the acceleration data is representative of acceleration of the shoulder, the rotation data is representative of rotation of the shoulder and/or the position data is representative of the position of the shoulder is provided to the motion monitor 107. Additionally or alternatively, during and/or throughout the swing, in some examples, the hip sensor 111 captures the acceleration of the hip, rotation of the hip and/or position of the hip. In some examples, the acceleration data is representative of acceleration of the hip, the rotation data is representative of rotation of the hip and/or the position data is representative of the position of the hip is provided to the motion monitor 107. In other words, the wrist sensor 108, the shoulder sensor 110 and the hip sensor 111 capture example motion data 122 during the swing that is provided to the motion monitor 107 for processing, analysis, etc. For sake of clarity, it is noted that although the examples refer to a hip sensor, a wrist sensor and a shoulder sensor, any or all of the hip sensor, the wrist sensor and/or the shoulder sensor may actually include more than one sensor. Additionally or alternatively, additional sensors may be used on other parts of the body (e.g., on joints of the body).

To enable further analytics to be performed on the motion data 122 and/or to enable the orientation of the respective sensors 108, 110, 111 to be determined throughout the swing, in the example of FIG. 1, the mobile device 104 includes an example motion data analyzer 127. While the example of FIG. 1 depicts the motion data analyzer 127 being implemented in the mobile device 104, some or all of the motion data analyzer 127 may be implemented at the remote facility 105. In some such examples, the remote facility 105 accesses the motion data 122 and/or the image/video data 124 from the wrist senor 108, the shoulder sensor 110 and/or the hip sensor 111 and/or from the mobile device 104.

Regardless whether the motion data analyzer 127 is implemented by and/or at the mobile device 104 or the remote facility 105, in some examples, the motion data analyzer 127 accesses the motion data 122 from the motion monitor 107 of the smart apparel 102 and fuses the acceleration data of the motion data 122 from one or more of the wrist sensor 108, the shoulder sensor 110 and/or hip sensor 111 with the rotation data and/or the position data of the motion data 122 from one or more of the wrist sensor 108, the shoulder sensor 110 and/or hip sensor 111. The motion data 122 may be fused using an inertial measurement unit algorithm and/or another fusion algorithm. In some examples, the motion data analyzer 127 performs analytics on the fused data and/or the individual components of the motion data 122 to identify posture-specific metrics, key performance indicators and/or metrics for a swing. However, the posture-specific metrics, the key performance indicators and/or the metrics may be specific to and/or associated with any movement-based activity being monitored.

The key performance indicators may include bio-kinetic feedback (e.g., full-body bio-kinetic feedback) and/or bio-kinetic performance indicators that focus on causes and/or coordinated movement of portions of the body (e.g., different joints of the body) relevant to the action being performed (e.g., throwing a football, hitting a baseball, etc.). In some examples, the key performance indicators are determined by characterizing a progression of a swing based on an angular velocity profile and/or how velocity peaks from the respective sensors 108, 110, 111 correspond and/or align with one another. For example, the progression of the swing may be analyzed by combining the motion data 122 from the hip sensor 111 and one or more of the shoulder sensor 110 and/or the wrist sensor 108 to determine how the hip is moving in relation to the shoulder and/or to determine the position of the hip relative to the shoulder at different phases and/or each phase of the swing.

In the example of FIG. 1, the mobile device 104 also includes an example display 128 that enables example image/video data 124 and/or associated results to be displayed and, thus, compared to a previous swing(s) and/or other historical data. The image/video data 124 may be captured by an example camera 126 of the mobile device 104. To enable the image/video data 124 to be viewed in association with the results of analytics performed on the motion data 122, in some examples, the key performance indicators and/or the results are shown overlaying and/or annotating the image/video data 124 on the display 128. Additionally or alternatively, in some examples, the example mobile device 104 enables telestration to be performed on the image/video data 124 on the display 128. While the above examples mention swinging a baseball bat and/or hitting a baseball as an example of a swing-based sport in which the example smart apparel system 100 can be implemented, the examples disclosed herein can be used in connection with in any other sport such as, for example, football, golf, tennis, swimming, baseball throwing/pitching, skiing, etc.

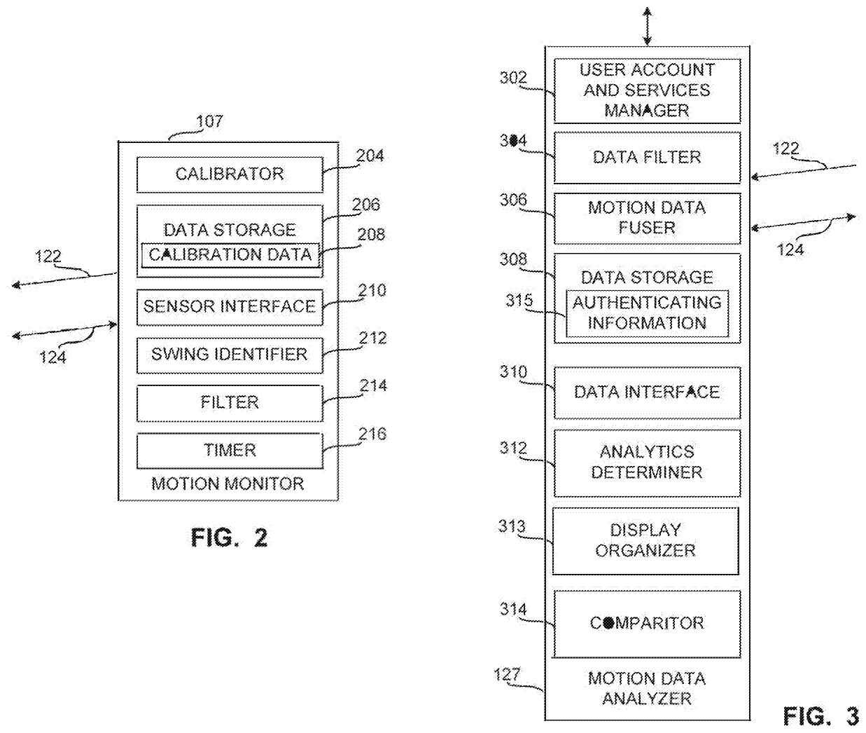

FIG. 2 illustrates an example implementation of the motion monitor 107 of FIG. 1. In the illustrated example, the motion monitor 107 includes an example calibrator 204, example data storage 206 including calibration data 208 and reference motion data 2090, an example sensor interface 210, an example swing identifier 212, an example filter 214 and an example timer 216.

To accurately monitor movement of the motion sensors 114, 116 and/or 118, in some examples, the calibrator 204 applies the calibration data 208 to the motion data 122 in real-time as the motion data 122 is being obtained and/or sampled to account for variances between the motion sensors 114, 116 and/or 118. For example, when the motion sensors 114, 116 and/or 118 are implemented as microelectromechanical systems (MEMS), the calibration data 208 may account for per-unit differences (e.g., mechanical differences). In some examples, the calibration data 208 is downloaded to and/or otherwise obtained for storage at the data storage 206 prior to, while and/or after the smart apparel 102 is being manufactured and/or otherwise produced in accordance with the teachings of this disclosure.

To identify swing data and/or non-swing data within the motion data 122, in some examples, the swing identifier 212 compares the motion data 122 to reference motion data 209 stored in the data storage 206. For example, the swing identifier 212 can compare the wrist acceleration and speed represented in the motion data 122, the shoulder acceleration and speed represented in the motion data 122 and/or the shoulder rotation speed represented in the motion data 122 to the reference motion data 209 to identify when a swing has occurred. In some examples, the reference motion data 209 is downloaded to and/or otherwise obtained for storage at the data storage 206 prior to, while and/or after the smart apparel 102 is being manufactured and/or otherwise produced in accordance with the teachings of this disclosure. The reference motion data 209 may include motion data associated with a swing including associated times that different actions are to occur and/or the coordinated movements that are indicative of a swing. In some examples, the reference motion data 209 may include motion data not associated with a swing (e.g., non-swing motion data). In some examples, non-swing motion data reflects movement of the wrists but does not include motion data reflecting movement of the shoulder and/or hips.

Once identified, in some examples, the filter 214 removes the non-swing data from the motion data 122. When a swing is identified, in some examples, the timer 216 determines an amount of time taken during different portions of the swing. For example, when the swing is identified as occurring, the timer 216 determines a start time and an end time associated with movement of the wrist sensor 108. Additionally or alternatively, when the swing is identified as occurring, the timer 216 determines a start time and an end time associated with movement of the shoulder sensor 110. Additionally or alternatively, when the swing is identified as occurring, the timer 216 determines a start time and an end time associated with movement of the hip sensor 111. To enable the motion data 122 to be accessed by the mobile device 104 and/or the remote facility 105 for further processing and/or storage, in the illustrated example, the motion monitor 107 includes the example sensor interface 210. The sensor interface 210 may include communication circuitry and supporting software/firmware to transmit and/or receive commands and/or data via any past, present or future communication protocol (e.g., cellular; Wi-Fi; and/or Bluetooth).

While an example manner of implementing the motion monitor 102 of FIG. 1 is illustrated in FIG. 2, one or more of the elements, processes and/or devices illustrated in FIG. 2 may be combined, divided, re-arranged, omitted, eliminated and/or implemented in any other way. Further, the example calibrator 204, the example data storage 206, the example sensor interface 210, the example swing identifier 212, the example filter 214, the example timer 216 and/or, more generally, the example motion monitor 107 of FIG. 2 may be implemented by hardware, software, firmware and/or any combination of hardware, software and/or firmware. Thus, for example, any of the example calibrator 204, the example data storage 206, the example sensor interface 210, the example swing identifier 212, the example filter 214, the example timer 216 and/or, more generally, the example motion monitor 107 of FIG. 2 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example calibrator 204, the example data storage 206, the example sensor interface 210, the example swing identifier 212, the example filter 214, the example timer 216 and/or, more generally, the example motion monitor 107 of FIG. 1 is/are hereby expressly defined to include a non-transitory computer readable storage device or storage disk such as a memory, a digital versatile disk (DVD), a compact disk (CD), a Blu-ray disk, etc. including the software and/or firmware. Further still, the example motion monitor 107 of FIG. 1 may include one or more elements, processes and/or devices in addition to, or instead of, those illustrated in FIG. 2, and/or may include more than one of any or all of the illustrated elements, processes and devices.

FIG. 3 illustrates an example implementation of the example motion data analyzer 127 of FIG. 1. In the illustrated example, the motion data analyzer 127 includes an example user account and services manager 302, an example data filter 304, an example motion data fuser 306, example data storage 308, an example data interface 310, an example analytics determiner 312, an example display organizer 313 and an example comparator 314.

In some examples, the user account and services manager 302 manages data associated with a user profile including authorizing access to the user profile based on account login information being received and authorized. The user profile and associated data may be stored in the data storage 308. In some examples, the user profile includes data associated with motion-based activities performed at different times. Additionally or alternatively, the user profile may include and/or organize data associated with a first motion based activity and/or swing in a structured format and/or organize data associated with a second motion based activity and/or swing in a structured format. Such data may include key performance indicators, metrics, image data, video data, etc., including, for example, historical motion data associated movement based activities such as, for example, hitting a baseball, etc.

In some examples, to enable a user account and/or profile to be accessed, the example user account and services manager 302 determines whether an account access request has been received (e.g., whether login information has been received) and, once received, if the login information authorizes access to the user profile. In some examples, the account access request and/or the profile login information are received at the data interface 310 and the profile login information is authenticated by the user account and services manager 302 comparing the login information received to authenticating information 315 stored at the data storage 308. However, authorization may be provided in any suitable way. For example, in examples in which the user account and services manager 302 is implemented at the remote facility 105, the login information may be authenticated by the motion data analyzer 127 of the mobile device 104 106 communicating with the remote facility 105 and the remote facility 105 providing the authentication.

In some examples, to enable processing and/or analytics to be performed on the motion data 122, the data interface 310 of the motion data analyzer 127 accesses the motion data 122 and the data filter 304 identifies noise present in the motion data 122. Once identified, the data filter 304 may filter the noise present within the motion data 122. The data filter 304 may be implemented as a low pass filter and the noise may not associated with motion. The data interface 310 may include communication circuitry and supporting software/firmware to transmit and/or receive commands and/or data via any past, present or future communication protocol (e.g., cellular; Wi-Fi; and/or Bluetooth).

To fuse the acceleration data of the motion data 122 from one or more of the wrist sensor 108, the shoulder sensor 110 and/or hip sensor 111 with the rotation data and/or the position data of the motion data 122 of the wrist sensor 108, the shoulder sensor 110 and/or hip sensor 111, in some examples, the motion data fuser 306 applies an inertial measurement unit algorithm and/or another fusion algorithm to the motion data 122. In some examples, fusing the motion data 122 includes the motion data fuser 306 combining the motion data 122 from the hip sensor 111 and one or more of the shoulder sensor 110 and/or the wrist sensor 108 to determine how the hip of the individual and/or athlete wearing the smart apparel 102 is moving in relation to the shoulder of the individual and/or athlete wearing the smart apparel 102. Additionally or alternatively, in some examples, fusing the motion data 122 includes the motion data fuser 306 combining the motion data 122 from the hip sensor 111 and one or more of the shoulder sensor 110 and/or the wrist sensor 108 to determine the position of the hip relative to the shoulder at each phase of the swing. In examples in which the motion data fuser 306 uses an inertial measurement unit algorithm to fuse the data, the inertial measurement unit algorithm may include a low pass filter to enable a first order integration to have a relatively smooth result with regard to speed.

To identify posture-specific metrics, key performance indicators and/or metrics for a swing, analytics are performed on the fused data and/or the motion data 122 at the mobile device 104 by the analytics determiner 312 accessing the fused data and/or the motion data 122 from the data storage 308 and processing and/or performing analytics on the fused data and/or the motion data 122. In some examples, the analysis includes the analytics determiner 312 determining kinematic motion for the wrist sensor 108, the shoulder sensor 110 and/or the hip sensor 111 including, for example, the speed and/or rotation at the respective sensors 108, 110 and/or 111 and/or the associated motion sensors 114, 116, 118.

In some examples, the metrics determined by the analytics determiner 312 include forward lean, torso flexion, shoulder and/or lateral tilt, hand path side view, hand path top view and/or maximum separation. The metrics determined by the analytics determiner 312 may include how the hip of an individual wearing the smart apparel 102 moves relative to the shoulder of the individual wearing the smart apparel 102. For example, the analytics determiner 312 can determine the speed that the hip and the shoulder move relative to one another and/or the orientation of the hip relative to the shoulder in different phases of the swing and/or any other monitored movement based activity.

In some examples, the key performance indicators include hip speed, hip rotation, shoulder speed, shoulder rotation, hand speed, hand rotation and/or shoulder dip. In some examples, to calculate and/or determine peak speeds of the different body components and/or joints of the body throughout the swing and/or to characterize the progression of the swing, the analytics determiner 312 identifies prominent velocity peaks within the motion data 122 and determines how the velocity peaks within the motion data 122 align with one another. Additionally or alternatively, in some examples, the analytics determiner 312 characterizes the progression of the swing based on a relative angular velocity profile.

To estimate the start and end times of the swing and/or a kinetic chain identifying the relative start and/or stop times for movement of the hips, movement of the shoulder and movement of the wrists, in some examples, the analytics determiner 312 analyses and/or otherwise processes the motion data 122 from the respective sensors 108, 110, 111. In some examples, the analytics determiner 312 determines the handedness of the swing (e.g., right handed batter versus left handed batter) based on the rotation direction of the motion data 122.

In some examples, to associate the image/video data 124 of a swing with the corresponding key performance indicators and/or metrics, the display organizer 313 accesses the image/video data 124 from the data storage 308 and/or the camera 126 and annotates, overlays and/or otherwise associates the key performance indicators and/or metrics with the associated image/video data 124 for display at, for example, the display 128 of the mobile device 104.

To enable historical data and/or movement based data to be compared, the motion analyzer 127 includes the comparator 314. In some examples, the comparator 314 accesses the image/video data 124 from different ones of the monitored motion based activities to perform a comparison of the data and/or to identify similarities and/or differences. Additionally or alternatively, in some examples, the comparator 314 accesses key performance indicators and/or metrics from different ones of the monitored motion based activities to perform a comparison of the data and/or to identify similarities and/or differences.

While an example manner of implementing the motion data analyzer 127 of FIG. 1 is illustrated in FIG. 3, one or more of the elements, processes and/or devices illustrated in FIG. 3 may be combined, divided, re-arranged, omitted, eliminated and/or implemented in any other way. Further, the example user account and services manager 302, the example data filter 304, the example motion data fuser 306, the example data storage 308, the example data interface 310, the example analytics determiner 312, the example display organizer 313, the example comparator 314 and/or, more generally, the example motion data analyzer 127 of FIG. 3 may be implemented by hardware, software, firmware and/or any combination of hardware, software and/or firmware. Thus, for example, any of the example user account and services manager 302, the example data filter 304, the example motion data fuser 306, the example data storage 308, the example data interface 310, the example analytics determiner 312, the example display organizer 313, the example comparator 314 and/or, more generally, the example motion data analyzer 127 of FIG. 3 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example user account and services manager 302, the example data filter 304, the example motion data fuser 306, the example data storage 308, the example data interface 310, the example analytics determiner 312, the example display organizer 313, the comparator 314 of FIG. 3 is/are hereby expressly defined to include a non-transitory computer readable storage device or storage disk such as a memory, a digital versatile disk (DVD), a compact disk (CD), a Blu-ray disk, etc. including the software and/or firmware. Further still, the example motion data analyzer 127 of FIG. 3 may include one or more elements, processes and/or devices in addition to, or instead of, those illustrated in FIG. 3, and/or may include more than one of any or all of the illustrated elements, processes and devices.

FIG. 4 illustrates an example smart apparel top 500, such as a shirt or jacket that can be used to implement the smart apparel 102 of FIG. 1. In the illustrated example, the smart apparel top 500 includes an example wrist sensor 504, an example shoulder sensor 506 and an example hip sensor 508 that are coupled together by an example wrapper 510. In some examples, the wrapper 510 is a TPE-based wrapper that deters ingress of fluid and/or debris (e.g., sweat ingress, water ingress, etc.) into the wrapper 510. The example wrist sensor 504 can be used to implement the wrist sensor 108 of FIG. 1. The example shoulder sensor 504 can be used to implement the shoulder sensor 110 of FIG. 1. The example hip sensor 508 can be used to implement the hip sensor 111 of FIG. 1.

To enable motion data accessed from the sensors 504, 506, 508 to be processed at the smart apparel top 500 and/or to be communicated to another device (e.g., the mobile device 104, the remote facility 105), the example motion monitor 107 as set forth herein may be housed adjacent at least one of the wrist sensor 504, the shoulder sensor 506 and/or the hip sensor 508. In some examples, the wrist sensor 504, the shoulder sensor 506 and/or the example hip sensor 508 are communicatively coupled using, for example, an inter-integrated circuit (I²C) protocol. However, any past, present or future communication protocol (e.g., cellular; Wi-Fi; and/or Bluetooth) may additionally or alternatively be used.

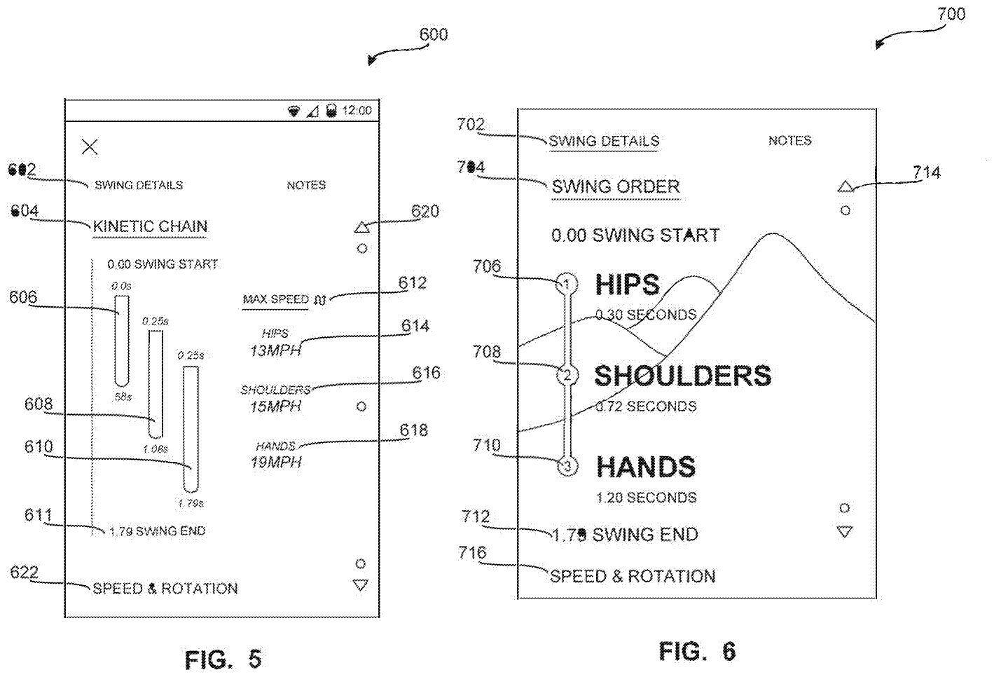

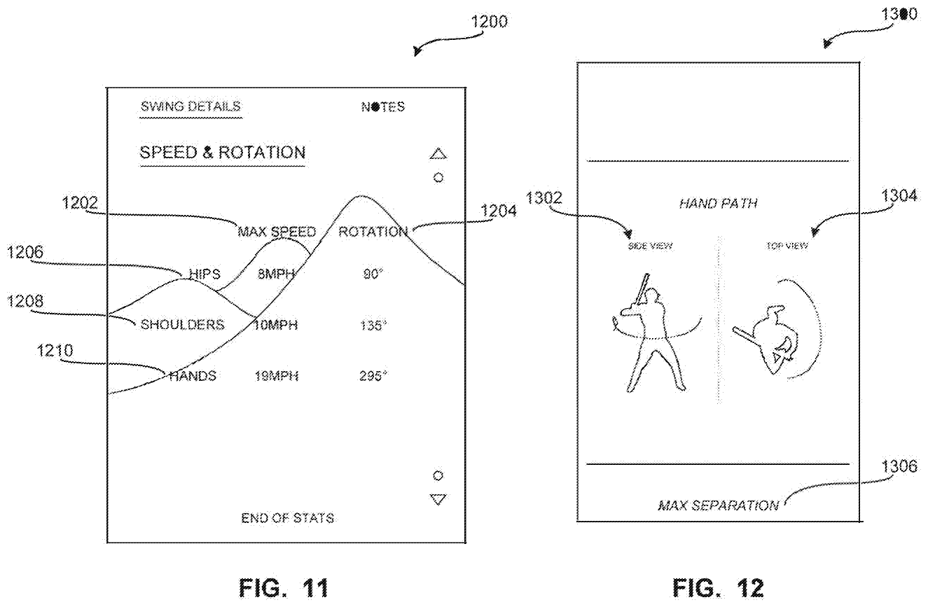

FIG. 5 is an example user interface 600 that can be displayed using the example display 128 of FIG. 1. In the illustrated example, the interface 600 includes swing details 602 including a kinetic chain 604 illustrating the relative start and stop times of movement of the hips 606, the shoulder 608 and the wrists 610, as well as a total swing time 611. Additionally, in the illustrated example, a max speed 612 is included for the hips 614, the shoulders 616 and the hands 618. In the example of FIG. 5, the user interface 600 includes a scroll bar 620 and an example speed & rotation heading 622 to enable a user to advance to a different user interface associated with speed & rotation (FIG. 11) should the example speed & rotation heading 622 be selected.

FIG. 6 is another example user interface 700 that can be displayed using the example display 128 of FIG. 1. In the illustrated example, the interface 700 includes swing details 702 including a swing order 704 illustrating the relative start times of the movement of the hips 706, the shoulders 708 and the wrists 710 as well as a total swing time 712. In the example of FIG. 6, the user interface 700 includes a scroll bar 714 and an example speed & rotation heading 716 to enable a user to advance to a different user interface associated with speed & rotation (FIG. 11) should the speed & rotation heading 716 be selected.

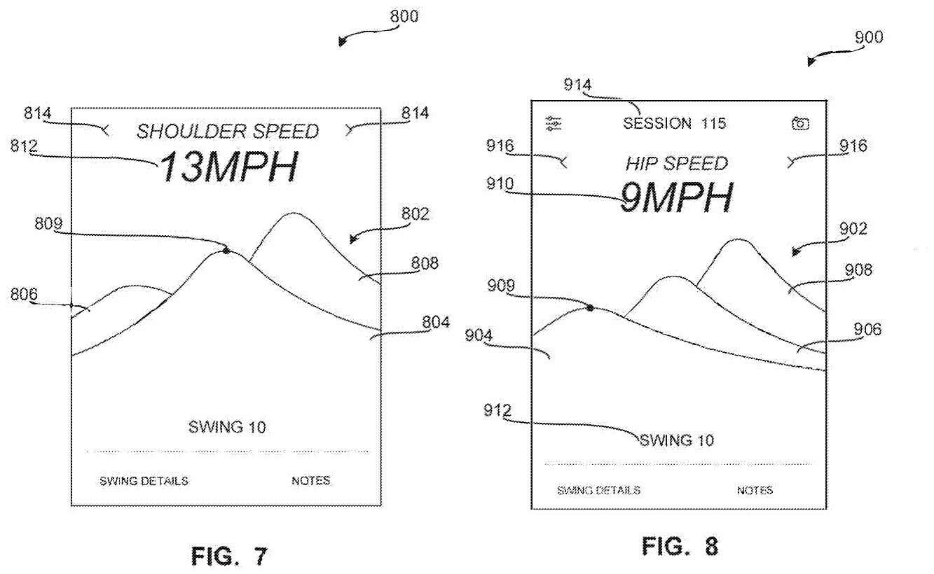

FIG. 7 is another example user interface 800 that can be displayed using the example display 128 of FIG. 1. In the illustrated example, the interface 800 includes a graph 802 representing a shoulder speed curve 804, a hip speed curve 806 and a hand speed curve 808. In this example, the shoulder speed curve 804 is in the forefront of the graph 802, an indicator 809 identifies the location of the maximum shoulder speed on the shoulder speed curve 804 and a value of the associated shoulder speed 812 is represented (e.g., 13 mph). Further, the user interface 800 includes the hip speed curve 806 and the hand speed curve 808 in the background of the graph 802. In the example of FIG. 7, the user interface 800 includes arrows 814 to enable a user to change to other user interfaces (FIGS. 8, 9) should the arrows 814 be selected.

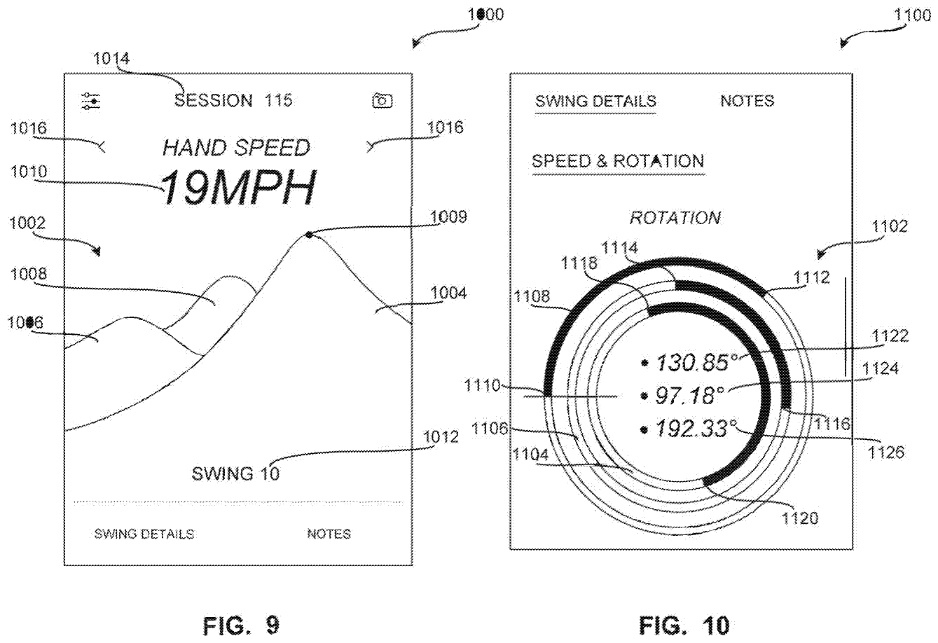

FIG. 8 is another example user interface 900 that can be displayed using the example display 128 of FIG. 1. In the illustrated example, the interface 900 includes a graph 902 representing a hip speed curve 904, a shoulder speed curve 906 and a hand speed curve 908. In this example, the hip speed curve 904 is in the forefront of the graph 902 and an indicator 909 is included on the hip speed curve 904 to identify a location of the maximum hip speed on the hip speed curve 904. Furthermore, a value of the maximum hip speed 910 (e.g., 9 mph), a swing number 912 and a session number 914 are included. In the example of FIG. 8, the user interface 900 includes arrows 916 to enable a user to change to other user interfaces (FIGS. 9, 10) should the arrows 916 be selected.

FIG. 9 is another example user interface 1000 that can be displayed using the example display 128 of FIG. 1. In the illustrated example, the interface 1000 includes a graph 1002 representing a hand speed curve 1004, a hip speed curve 1006 and a shoulder speed curve 1008. In this example, the hand speed curve 1004 is in the forefront of the graph 1002 and an indicator 1009 is included on the hand speed curve 1004 to identify a location of the maximum hand speed curve 1004. Further, a value representing a value of the maximum hand speed 1010 (e.g., 9 mph) and a swing number 1012 and a session number 1014 are included. In the example of FIG. 9, the user interface 1000 includes arrows 1016 to enable a user to change to other user interfaces (FIGS. 7, 8) should the arrows 1016 be selected.

FIG. 10 is another example user interface 1100 that can be displayed using the example display 128 of FIG. 1. In the illustrated example, the interface 1100 includes a concentric circle graph 1102 representing a shoulder rotation arc 1104, a hip rotation arc 1106 and a hand rotation arc 1108. In this example, the shoulder rotation arc 1104 includes a start 1110 and an end 1112, the hip rotation arc 1106 includes a start 1114 and an end 1116 and the hand rotation arc 1108 includes a start 1118 and an end 1120. As such, the concentric circle graph 1102 enables the rotation and the relative starts 1110, 1114, 1118 and ends 1112, 1116, 1120 of the shoulder rotation arc 1104, the hip rotation arc 1106 and the shoulder rotation arc 1108 to be compared and/or viewed. Further, in the illustrated example, shoulder rotation degrees of rotation 1122, hip rotation degrees of rotation 1124 and hand rotation degrees of rotation 1126 are represented within the concentric circle graph 1102.

FIG. 11 is another example user interface 1200 that can be displayed using the example display 128 of FIG. 1. In the illustrated example, the interface 1200 includes a first column 1202 for maximum speed and a second column 1204 for rotation throughout a baseball swing. As shown in the example of FIG. 11, metrics for hips 1206, shoulders 1208 and hands 1210 are included under the respective columns 1202, 1204.

FIG. 12 is another example user interface 1300 that can be displayed using the example display 128 of FIG. 1. In the illustrated example, the interface 1300 includes a side view graph 1302 representing a baseball swing and/or a hand path side view and a top view graph 1304 representing a baseball swing and/or a hand path top view. In some examples, the side view graph 1302 and the top view graph 1304 are generated based on the processing of the motion data 122. In this example, the user interface 1300 includes a maximum separation heading 1306 to enable the user interface 1300 to advance to a different user interface associated with maximum separation should the max separation heading 1306 be selected.

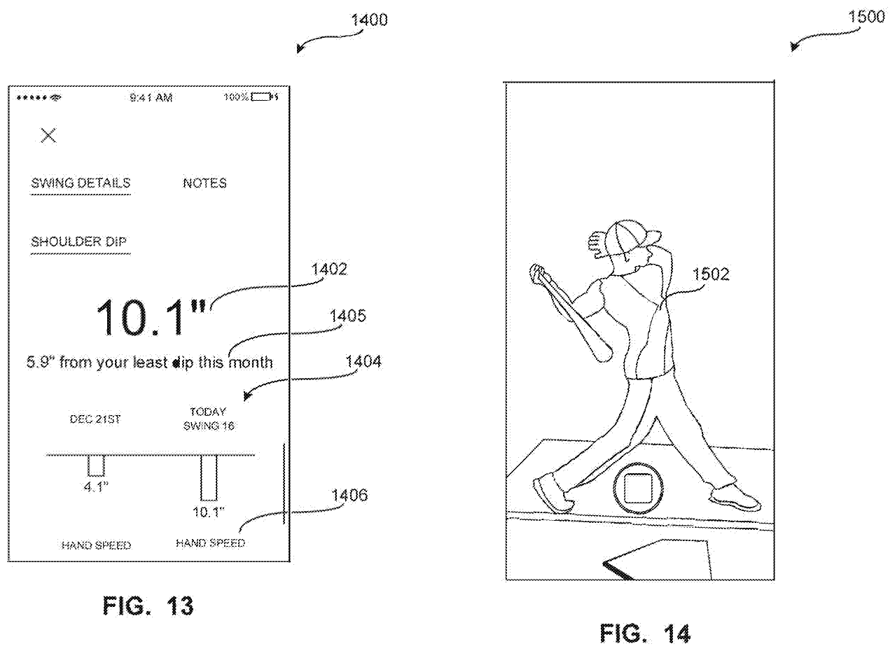

FIG. 13 is another example user interface 1400 that can be displayed using the example display 128 of FIG. 1. In the illustrated example, the user interface 1400 includes shoulder dip details 1402 and a graphical comparison 1404 between historical shoulder dip details. As shown, in the illustrated example, on a prior day (e.g., December 21), the shoulder dip was determined to be 4.1 inches and the shoulder dip of the current day (e.g., June 19) was determined to be 10.1 inches. Further, in this example, the user interface 1400 displays a difference 1405 between previous and current shoulder dips and/or swings. In this example, the user interface 1400 includes a hand speed heading 1406 to enable a user to advance to a different user interface associated with hand speed should the hand speed heading 1406 be selected.

FIG. 14 illustrates another example user interface 1500 of image and/or video data of an individual 1502 hitting a baseball captured and/or obtained by the camera 126.

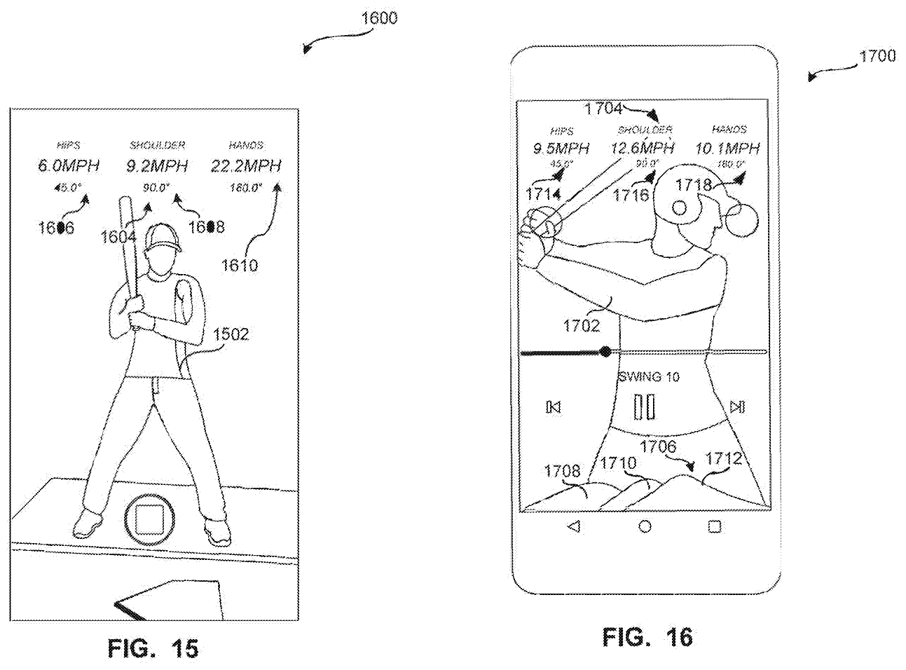

FIG. 15 illustrates another example user interface 1600 representing the individual 1502 and metrics 1604 annotating and/or overlaying the image and/or video data. In the example of FIG. 15, the metrics 1604 include hip speed and rotation 1606, shoulder speed and rotation 1608 and hand speed and rotation 1610. While the user interface 1600 includes some metrics annotating and/or overlaying the image and/or video data, other metrics and/or key performance indicators may be included in other examples.

FIG. 16 illustrates another example user interface 1700 representing an individual 1702 hitting a baseball and metrics 1704 and speed curves 1706 annotating and/or overlaying the image and/or video data of the individual 1702. In the example of FIG. 16, the speed curves 1706 include a first speed curve 1708 associated with hip speed, a second speed curve 1710 associated with shoulder speed and a third speed curve 1712 associated with hand speed. In this example, the metrics 1704 include hip speed and rotation 1714, shoulder speed and rotation 1716 and hand speed and rotation 1718. While the user interface 1700 includes some metrics annotating and/or overlaying the image and/or video data, other metrics and/or key performance indicators may be included in other examples.

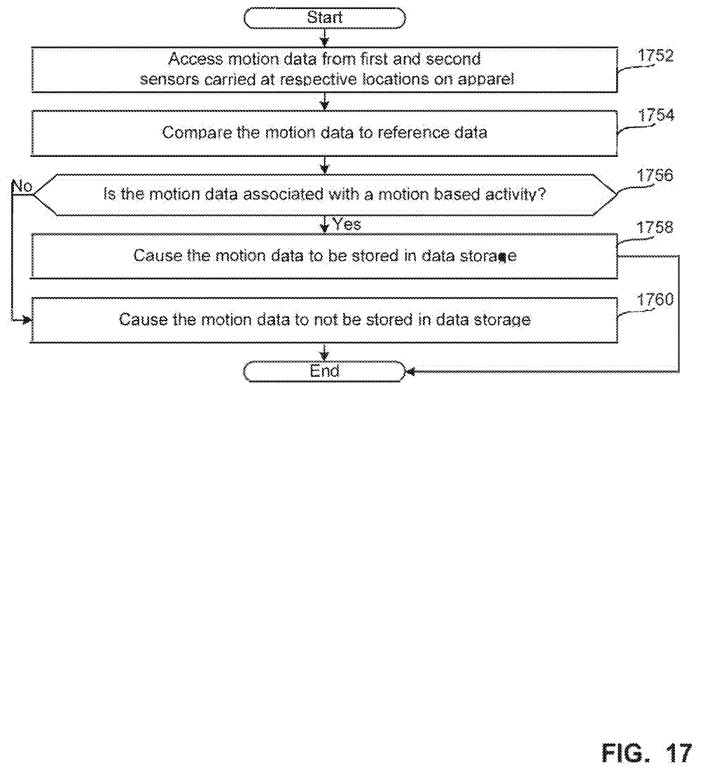

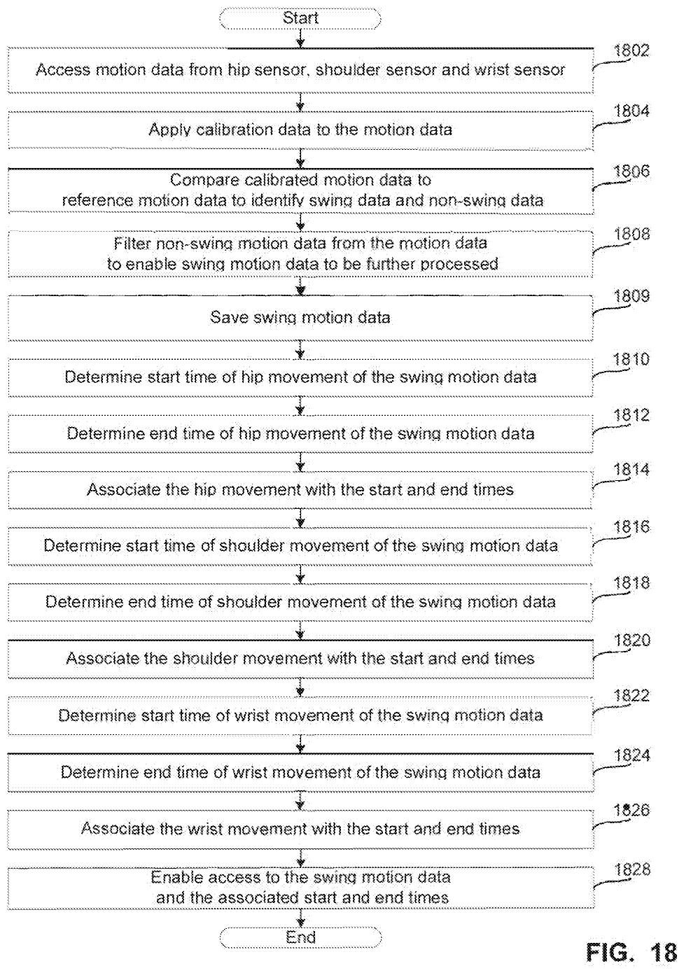

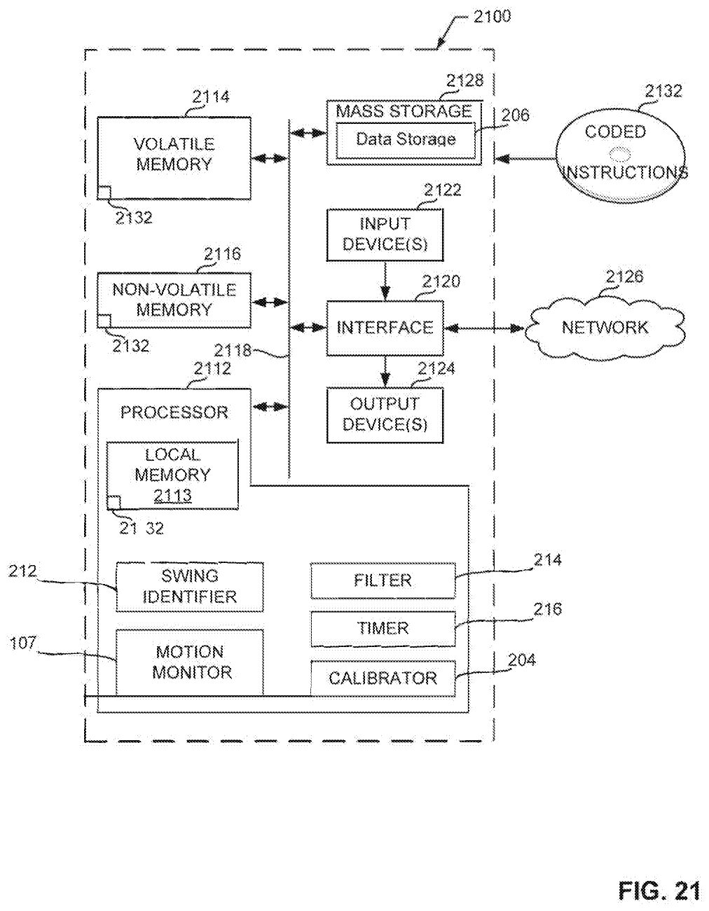

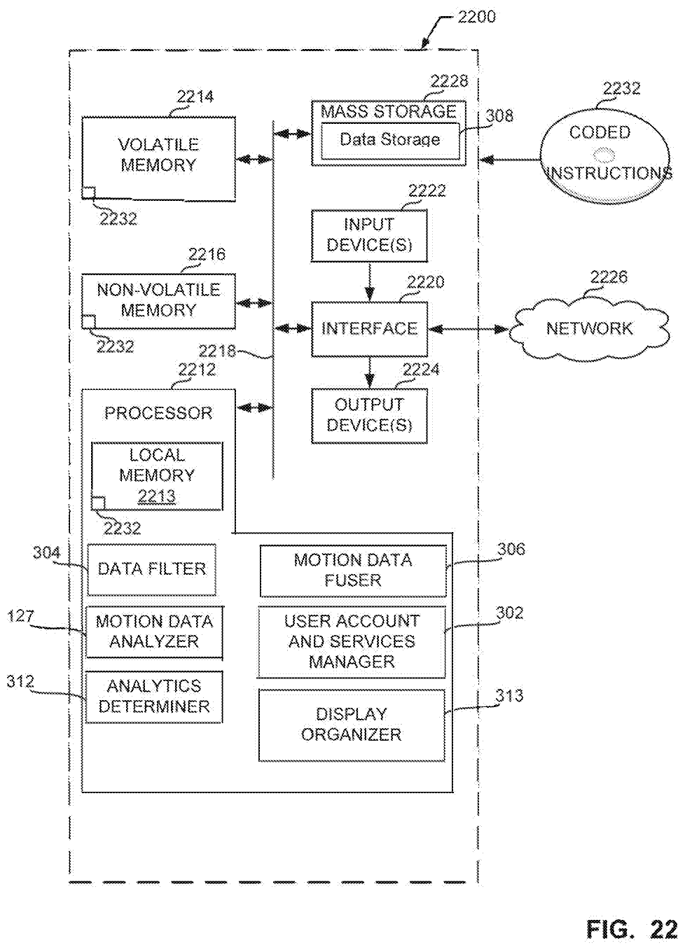

Flowcharts representative of example machine readable instructions for implementing the motion monitor 107 and the motion data analyzer 127 of FIGS. 2 and 3 are shown in FIGS. 17, 18, 19 and 20. In this example, the machine readable instructions comprise a program for execution by a processor such as the processors 2112 and 2212 shown in the example processor platforms 2100 and 2200 discussed below in connection with FIGS. 21 and 22. The program may be embodied in software stored on a non-transitory computer readable storage medium such as a CD-ROM, a floppy disk, a hard drive, a digital versatile disk (DVD), a Blu-ray disk, or a memory associated with the processors 2112 and 2212, but the entire program and/or parts thereof could alternatively be executed by a device other than the processors 2112 and 2212 and/or embodied in firmware or dedicated hardware. Further, although the example program is described with reference to the flowcharts illustrated in FIGS. 17, 18, 19 and 20, many other methods of implementing the motion monitor 107 and the motion data analyzer 127 of FIGS. 2 and 3 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, a Field Programmable Gate Array (FPGA), an Application Specific Integrated circuit (ASIC), a comparator, an operational-amplifier (op-amp), a logic circuit, etc.) structured to perform the corresponding operation without executing software or firmware.

As mentioned above, the example processes of FIGS. 17, 18, 19 and 20 may be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. “Including” and “comprising” (and all forms and tenses thereof) are used herein to be open ended terms. Thus, whenever a claim lists anything following any form of “include” or “comprise” (e.g., comprises, includes, comprising, including, etc.), it is to be understood that additional elements, terms, etc. may be present without falling outside the scope of the corresponding claim. As used herein, when the phrase “at least” is used as the transition term in a preamble of a claim, it is open-ended in the same manner as the term “comprising” and “including” are open ended.

The program of FIG. 17, which may be executed to implement the motion monitor 107, begins at block 1752 with the motion monitor 107 accessing the motion data 122 from two or more of the motion sensors 114, 116, 118 carried at respective locations on the smart apparel (block 1752). The swing identifier 212 compares the motion data 122 to reference motion data associated with a motion based activity of interest (e.g., a swing based activity) (block 1754). At block 1756, the swing identifier 212 determines if the motion data 122 is associated with the motion based activity corresponding to the reference motion data 209 (block 1756). Some motion based activities include football, basketball, baseball, soccer, tennis, bowling, etc., or, more generally, any movement based activities where interrelationships of body movements affect an outcome (e.g., throwing a curve ball, getting a strike in bowling, etc.).

If the motion data is associated with the motion based activity, the filter 214 does not filter the motion data 122 and the motion data 122 is stored in the data storage 206 (block 1758). If the motion data is not associated with the motion based activity, the filter 214 filters the motion data 122 and the motion data 122 is not stored in the data storage 206 (block 1760).

The program of FIG. 18, which may be executed to implement the example motion monitor 107 begins at block 1802 with the motion monitor 107 accessing the motion data 122 from the motion sensors 114, 116, 118 (block 1802). To enable the motion data to accurately reflect movement of the respective motion sensors 114, 116, 118, the calibrator 204 applies the calibration data 208 to the motion data 122 (block 1804). To differentiate between swing motion data and/or non-swing motion data within the motion data 122, the swing identifier 212 compares the motion data 122 to reference motion data (block 1806). Once identified, the filter 214 removes the non-swing motion data from the motion data 122 to enable the swing motion data to be further processed (block 1808). At block 1809, the swing motion data is stored at the data storage 206 (block 1809).

When processing the swing motion data, the motion monitor 107 triggers and/or causes the timer 216 to determine a start time (block 1810) and an end time (block 1812) of hip movement reflected within the swing motion data. At block 1814, the motion monitor 107 associates the start and stop times with the hip movement. Further, when processing the swing motion data, the motion monitor 107 triggers and/or causes the timer 216 to determine a start time (block 1816) and an end time (block 1818) of shoulder movement reflected within the swing motion data. At block 1820, the motion monitor 107 associates the start and stop times with the shoulder movement. Further, when processing the swing motion data, the motion monitor 107 triggers the timer 216 to determine a start time (block 1822) and an end time (block 1824) of wrist movement reflected within the swing motion data. At block 1826, the motion monitor 107 associates the start and stop times with the wrist movement. At block 1828, the sensor interface 210 enables the mobile device 104 and/or the remote facility 105 to access to the swing motion data 122 and the associated start and stop times, for example.

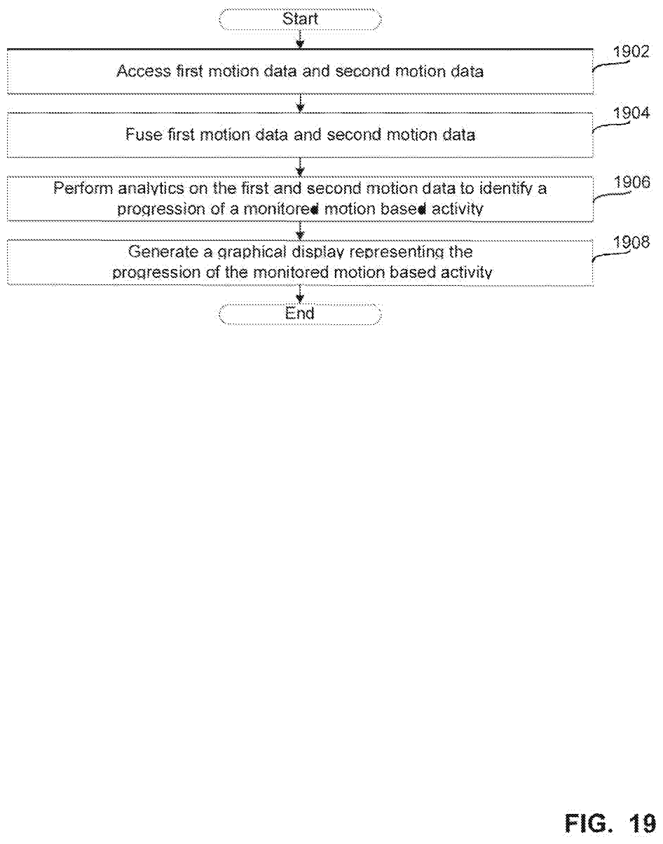

The program of FIG. 19, which may be executed to implement the example motion data analyzer 127, begins at block 1902 with the motion data analyzer 127 accessing first motion data 122 and second motion data 122 (block 1902). In some examples, the first motion data 122 is representative of motion of a first joint of a body of an individual wearing the smart apparel 102 and the second motion data 122 is representative of motion of a second joint of the body of the individual wearing the smart apparel 102. The motion data fuser 306 applies an inertial measurement unit algorithm and/or another fusion algorithm to the first and second motion data 122 to fuse the first and second motion data (block 1904). The analytics determiner 312 accesses the fused data and/or the motion data 122 from the data storage 308 and processes and/or performs analytics on the fused data and/or individual components of the motion data 122 to identify a progression of a monitored motion based activity (block 1906). The display organizer 313 generates a graphical display representing the progression of the monitored motion based activity (block 1908).

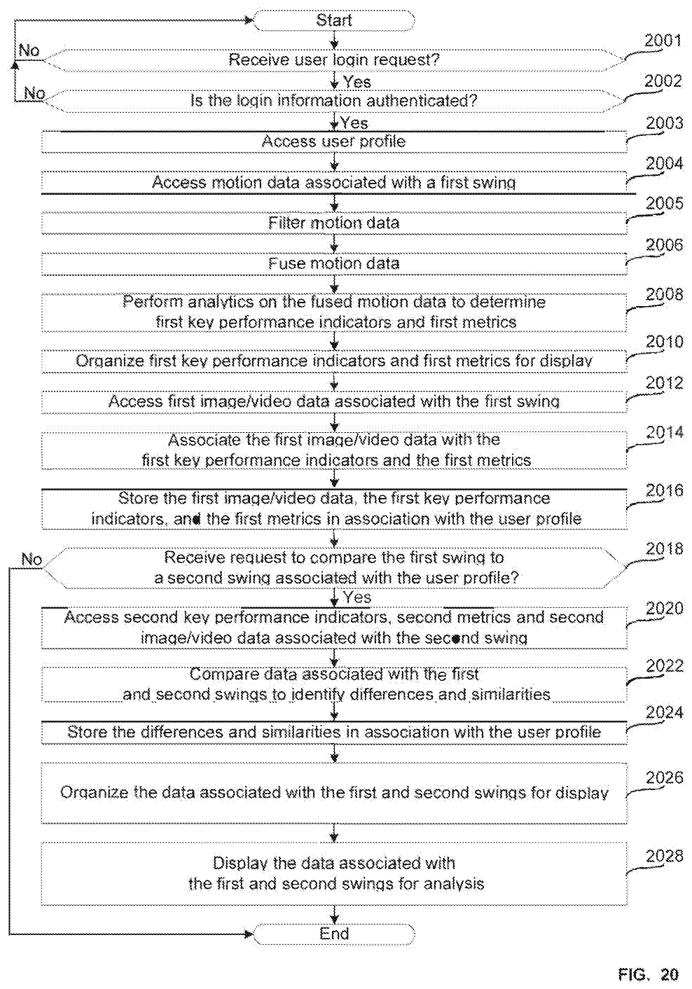

The program of FIG. 20, which may executed to implement the motion data analyzer 127, begins at block 2001 with the user account and services manager 302 determining whether a user login request has been received (block 2001). If a user login request has been received, the user account and services manager 302 determines whether login information has been received and, if so, if the login information has been authenticated by, for example, comparing the information received to the authenticating information 315 (block 2002). If the login information authorizes access to the user profile and/or if authorization has been granted to access the user profile, the user account and services manager 302 enables access to the user profile (block 2003).

At block 2004, the data interface 310 of the motion data analyzer 127 accesses the motion data 122 associated with a first swing (block 2004). The data filter 304 filters noise present in the motion data 122 (block 2005). In some examples, the motion data analyzer 127 identifies and/or characterizes noise (e.g., white noise) present in the motion data 122 not associated with motion prior the data filter 304 performing a filtering operation.

The motion data fuser 306 applies an inertial measurement unit algorithm and/or another fusion algorithm to the motion data 122 to fuse the acceleration data of the motion data 122 with the rotation data and/or the position data of the motion data 122 from one or more of the wrist sensor 108, the shoulder sensor 110 and/or hip sensor 111 (block 2006).

To identify posture-specific metrics, first key performance indicators and/or first metrics for the first swing, the analytics determiner 312 accesses the fused data and/or the motion data 122 from the data storage 308 and processes and/or performs analytics on the fused data and/or the motion data 122 (block 2008). In some examples, the analysis includes the analytics determiner 312 determining kinematic motion for the wrist sensor 108, the shoulder sensor 110 and/or the hip sensor 111 such as, for example, the speed and/or rotation at the respective sensors 108, 110 and/or 111 and/or the associated motion sensors 114, 116, 118. At block 2010, the display organizer 313 organizes the first key performance indicators and the first metrics for display (block 2010). For example, the display organizer 313 may map the identified key performance indicators and/or first metrics to a template and/or other data structure associated with the user profile.

To enable the image/video data 124 to be associated with the determined first key performance indicators and/or the first metrics, the display organizer 313 accesses the image/video data 124 associated with the first swing from the camera 126 and/or the data storage 308 (block 2012) and associates the first image/video data 124 with the first key performance indicators and/or the first metrics for storage, display and/or later analysis (block 2014). At block 2016, the motion data analyzer 127 stores the first image/video data, the first key performance indicators and the first metrics in association with a user profile at the data storage 308 and/or enables the remote facility 105 access to the first image/video data, the first key performance indicators and the first metrics for storage, etc. (block 2016). In some examples, storing the first image/video data, the first key performance indicators and the first metrics in association with a user profile includes the display organizer 313 mapping data associated with the first swing to one or more templates and/or other data structure associated with the user profile.

The data interface 310 determines whether a request has been received to compare the first swing to a second swing associated with the user profile (block 2018). If the data interface 310 receives a request to compare the first and second swings, the data interface 310 accesses data associated with the second swing from the data storage 308 (block 2020). The data associated with the second swing may include second key performance indicators, second metrics and/or second image/video data associated with the second swing.

The comparator 314 compares the data associated with the first swing to data associated with the second swing to identify similarities and/or differences (block 2022). The comparator 314 stores the similarities and/or differences in association with the user profile at the data storage 308 and/or enables the remote facility 105 access the data for storage, etc. (block 2024). The similarities and/or differences may be mapped by the display organizer 313 to a template and/or other data structure associated with the user profile by the display organizer 313.

The display organizer 313 organizes the data associated with first and second swings for display (block 2026). In some examples, the data includes the key performance indicators, the metrics, the image/video data and/or any data determined when comparing the first and second swings. At block 2028, the display organizer 313 causes the display 128 to display the data associated with the first and second swings for analysis, etc. (block 2028).

FIG. 21 is a block diagram of an example processor platform 2100 structured to execute the instructions of FIGS. 17 and 18 to implement the motion monitor 107 of FIG. 2. The processor platform 2100 can be, for example, a server, a personal computer, a mobile device (e.g., a cell phone, a smart phone, a tablet such as an iPad™), a personal digital assistant (PDA), an Internet appliance, or any other type of computing device.

The processor platform 2100 of the illustrated example includes a processor 2112. The processor 2112 of the illustrated example is hardware. For example, the processor 2112 can be implemented by one or more integrated circuits, logic circuits, microprocessors or controllers from any desired family or manufacturer. The hardware processor may be a semiconductor based (e.g., silicon based) device. In this example, the processor 2112 implements the calibrator 204, the swing identifier 212, the filter 214, the timer 216, and the motion monitor 107.

The processor 2112 of the illustrated example includes a local memory 2113 (e.g., a cache). The processor 2112 of the illustrated example is in communication with a main memory including a volatile memory 2114 and a non-volatile memory 2116 via a bus 2118. The volatile memory 2114 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 2116 may be implemented by flash memory and/or any other desired type of memory device. Access to the main memory 2114, 2116 is controlled by a memory controller.

The processor platform 2100 of the illustrated example also includes an interface circuit 2120. The interface circuit 2120 may be implemented by any type of interface standard, such as an Ethernet interface, a universal serial bus (USB), and/or a PCI express interface.

In the illustrated example, one or more input devices 2122 are included as an implementation of the sensor interface 210 of FIG. 2. In this example, the one or more input devices 2122 are connected to the interface circuit 2120. The input device(s) 2122 permit(s) a user to enter data and/or commands into the processor 2112. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 2124 are also included as an implementation of the sensor interface 210 of FIG. 2. In this example, the one or more output devices 2124 are connected to the interface circuit 2120 of the illustrated example. The output devices 2124 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 2120 of the illustrated example, thus, typically includes a graphics driver card, a graphics driver chip and/or a graphics driver processor.

The interface circuit 2120 of the illustrated example also includes a communication device such as a transmitter, a receiver, a transceiver, a modem and/or network interface card to facilitate exchange of data with external machines (e.g., computing devices of any kind) via a network 2126 (e.g., an Ethernet connection, a digital subscriber line (DSL), a telephone line, coaxial cable, a cellular telephone system, etc.).

The processor platform 2100 of the illustrated example also includes one or more mass storage devices 2128 for storing software and/or data. Examples of such mass storage devices 2128 include floppy disk drives, hard drive disks, compact disk drives, Blu-ray disk drives, RAID systems, and digital versatile disk (DVD) drives. In this example, the mass storage devices 2128 implements the data storage 206.

The coded instructions 2132 of FIGS. 17 and 18 may be stored in the mass storage device 2128, in the volatile memory 2114, in the non-volatile memory 2116, and/or on a removable tangible computer readable storage medium such as a CD or DVD.

FIG. 22 is a block diagram of an example processor platform 2200 structured to execute the instructions of FIGS. 19 and 20 to implement the motion data analyzer 127 of FIG. 3. The processor platform 2200 can be, for example, a server, a personal computer, a mobile device (e.g., a cell phone, a smart phone, a tablet such as an iPad™), a personal digital assistant (PDA), an Internet appliance, or any other type of computing device.

The processor platform 2200 of the illustrated example includes a processor 2212. The processor 2212 of the illustrated example is hardware. For example, the processor 2212 can be implemented by one or more integrated circuits, logic circuits, microprocessors or controllers from any desired family or manufacturer. The hardware processor may be a semiconductor based (e.g., silicon based) device. In this example, the processor 2212 implements the user account and services manager 302, the data filter 304, the motion data fuser 306, the analytics determiner 312, the display organizer 313 and the motion data analyzer 127.

The processor 2212 of the illustrated example includes a local memory 2213 (e.g., a cache). The processor 2212 of the illustrated example is in communication with a main memory including a volatile memory 2214 and a non-volatile memory 2216 via a bus 2218. The volatile memory 2214 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 2216 may be implemented by flash memory and/or any other desired type of memory device. Access to the main memory 2214, 2216 is controlled by a memory controller.

The processor platform 2200 of the illustrated example also includes an interface circuit 2220. The interface circuit 2220 may be implemented by any type of interface standard, such as an Ethernet interface, a universal serial bus (USB), and/or a PCI express interface.

In the illustrated example, one or more input devices 2222 are included as an implementation of the data interface 310 of FIG. 3. In this example, the one or more input devices 2222 are connected to the interface circuit 2220. The input device(s) 2222 permit(s) a user to enter data and/or commands into the processor 2212. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 2224 are also included as an implementation of the data interface 310 of FIG. 3. In the illustrated example, the output devices 2224 are connected to the interface circuit 2220 of the illustrated example. The output devices 2224 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 2220 of the illustrated example, thus, typically includes a graphics driver card, a graphics driver chip and/or a graphics driver processor.

The interface circuit 2220 of the illustrated example also includes a communication device such as a transmitter, a receiver, a transceiver, a modem and/or network interface card to facilitate exchange of data with external machines (e.g., computing devices of any kind) via a network 2226 (e.g., an Ethernet connection, a digital subscriber line (DSL), a telephone line, coaxial cable, a cellular telephone system, etc.).

The processor platform 2200 of the illustrated example also includes one or more mass storage devices 2228 for storing software and/or data. Examples of such mass storage devices 2228 include floppy disk drives, hard drive disks, compact disk drives, Blu-ray disk drives, RAID systems, and digital versatile disk (DVD) drives.

The coded instructions 2232 of FIGS. 19 and 20 may be stored in the mass storage device 2228, in the volatile memory 2214, in the non-volatile memory 2216, and/or on a removable tangible computer readable storage medium such as a CD or DVD.

From the foregoing, it will be appreciated that example methods, apparatus and articles of manufacture have been disclosed that enable analytics to be performed on swing-based sports and/or throw-based sports and, more generally, movement based on activities. In some examples, smart apparel is implemented with sensors at different points on (e.g., joints) of the body to enable motion data to be obtained. The smart apparel may be configured for use in football, basketball, soccer, tennis, bowling, etc., or, more generally, any movement based activities where interrelationships of body movements affect an outcome (e.g., throwing a curve ball, getting a strike in bowling, etc.).

### EXAMPLE 1

An example apparatus for apparel, the apparatus comprising, includes: a first sensor to be carried at a first location on the apparel to capture first motion data associated with a first part of a body wearing the apparel, a second sensor to be carried at a second location on the apparel and positioned to capture second motion data associated with a second part of the body; and a motion monitor to: compare at least one of the first motion data and the second motion data to reference data to determine when the first and second motion data are associated with a motion based activity; and cause the first and second motion data to be stored in data storage when the first and second motion data are associated with the motion based activity but not when the first and second motion data are not associated with the motion based activity.

### EXAMPLE 2

In Example 1 or other examples, the first part of the body is a first joint of the body and the second part of the body is a second joint of the body.

### EXAMPLE 3

In Examples 1, 2 or other examples, the apparatus includes a third sensor carried on the apparel at a third location to capture third motion data.

### EXAMPLE 4

In Example 3 or other examples, the motion monitor is to further compare the third motion data to the reference data to determine when the third motion data is associated with the motion based activity.

### EXAMPLE 5

In Examples 1, 2, 3, 4 or other examples, the motion based activity includes hitting a baseball.

### EXAMPLE 6

In Examples 1, 2, 3, 4, 5 or other examples, the first sensor and the second sensor are communicatively coupled.

### EXAMPLE 7

In Example 6 or other examples, the first sensor is communicatively coupled to the second sensor via a thermoplastic-based wrapper.

### EXAMPLE 8

In Examples 1, 2, 3, 4, 5, 6, 7 or other examples, the apparel includes a smart apparel.

### EXAMPLE 9

In Example 8 or other examples, the first location is one of a wrist area of the smart apparel, a shoulder area of the smart apparel, or a hip area of the smart apparel, and the second location is another one of the wrist area of the smart apparel, a shoulder area of the smart apparel, or a hip area of the smart apparel.

### EXAMPLE 10

In Examples, 1, 2, 3, 4, 5, 6, 7, 8, 9 or other examples, the motion monitor includes a sensor interface to communicate the first motion data and the second motion data to another device remote from the apparel.

### EXAMPLE 11

In Example 10 or other examples, the other device includes a mobile device.

### EXAMPLE 12

In Examples 1, 2, 3, 4, 5, 6, 7, 8, 9, 10, 11 or other examples, the data storage further includes first calibration data associated with the first sensor and second calibration data associated with second data, the motion monitor to apply the first calibration data to the first motion data and to apply second calibration data to the second motion data.

### EXAMPLE 13

In Examples 1, 2, 3, 4, 5, 6, 7, 8, 9, 10, 11, 12 or other examples, the first sensor includes an accelerometer or a gyroscope.

### EXAMPLE 14

In Examples 1, 2, 3, 4, 5, 6, 7, 8, 9, 10, 11, 12, 13 or other examples, the first motion data includes first acceleration data reflective of acceleration associated with the first location, first rotation data reflective of rotation associated with the first location, and first position data reflective of a position of the first location during the motion based activity.

### EXAMPLE 15

An example method, includes: comparing, by executing an instruction with at least one processor, at least one of first motion data and second motion data to reference data to determine when the first and second motion data are associated with a motion based activity, the first motion data associated with a first part of a body wearing apparel, the second motion data associated with a second part of the body wearing the apparel; and causing, by executing an instruction with the at least one processor, the first and second motion data to be stored in data storage when the first and second motion data are associated with the motion based activity but not when the first and second motion data are not associated with the motion based activity.

### EXAMPLE 16

In Example 15 or other examples, the first part of the body is a first joint of the body and the second part of the body is a second joint of the body.

### EXAMPLE 17

In Examples 15, 16 or other examples, the method includes comparing third motion data to the reference data to determine when the third motion data is associated with the motion based activity, the third motion data associated with a third part of the body wearing the apparel.

### EXAMPLE 18

In Examples 15, 16, 17 or other examples, the motion based activity includes hitting a baseball.

### EXAMPLE 19

In Examples 15, 16, 17, 18 or other examples, the apparel includes a smart apparel.

### EXAMPLE 20

In Examples 15, 16, 17, 18, 19 or other examples, the first location is one of a wrist area of the smart apparel, a shoulder area of the smart apparel, or a hip area of the smart apparel, and the second location is another one of the wrist area of the smart apparel, a shoulder area of the smart apparel, or a hip area of the smart apparel.

### EXAMPLE 21

In Examples 15, 16, 17, 18, 19, 20 or other examples, the method includes applying, by executing an instruction with the at least one processor, the first calibration data to the first motion data and applying second calibration data to the second motion data.

### EXAMPLE 22

In Examples 15, 16, 17, 18, 19, 20, 21 or other examples, the first motion data includes first acceleration data reflective of acceleration associated with the first location, first rotation data reflective of rotation associated with the first location, and first position data reflective of a position of the first location during the motion based activity.

### EXAMPLE 23

An example tangible computer-readable medium comprising instructions that, when executed, cause a processor to, at least: compare at least one of first motion data and second motion data to reference data to determine when the first and second motion data are associated with a motion based activity, the first motion data associated with a first part of a body wearing apparel, the second motion data associated with a second part of the body wearing the apparel; and cause the first and second motion data to be stored in data storage when the first and second motion data are associated with the motion based activity but not when the first and second motion data are not associated with the motion based activity.

### EXAMPLE 24

In Example 23 or other examples, the first part of the body is a first joint of the body and the second part of the body is a second joint of the body.

### EXAMPLE 25

In Examples 23, 24 or other examples, the instructions, when executed, cause the processor to compare third motion data to the reference data to determine when the third motion data is associated with the motion based activity, the third motion data associated with a third part of the body wearing the apparel.

### EXAMPLE 26

In Examples 23, 24, 25 or other examples the motion based activity includes hitting a baseball.

### EXAMPLE 27

In Examples 23, 24, 25, 26 or other examples, the instructions, when executed, cause the processor to apply first calibration data to the first motion data and to apply second calibration data to the second motion data.

### EXAMPLE 28

An example system for use with apparel, comprising: means for comparing at least one of first motion data and second motion data to reference data to determine when the first and second motion data are associated with a motion based activity, the first motion data associated with a first part of a body wearing apparel, the second motion data associated with a second part of the body wearing the apparel; and means for causing the first and second motion data to be stored in data storage when the first and second motion data are associated with the motion based activity but not when the first and second motion data are not associated with the motion based activity.

### EXAMPLE 29

In Example 28 or other examples, the first part of the body is a first joint of the body and the second part of the body is a second joint of the body.

### EXAMPLE 30

In Examples 28, 29 or other examples, the system includes means for comparing third motion data to the reference data to determine when the third motion data is associated with the motion based activity, the third motion data associated with a third part of the body wearing the apparel.

### EXAMPLE 31

In Examples 29, 30, 31 or other examples, the motion based activity includes hitting a baseball.

### EXAMPLE 32

In Examples 29, 30, 31, 32 or other examples, the system includes means for applying first calibration data to the first motion data and applying second calibration data to the second motion data.

### EXAMPLE 33

An example apparatus, includes: a data interface to access first motion data and second motion data generated by the smart apparel, the first motion data associated with a first joint on a body and the second motion data associated with a second joint on the body; a motion data fuser to fuse the first motion data and the second motion data; an analytics determiner to process the fused first and second motion data to identify a progression of a motion based activity; and a display organizer to generate a graphical display representing the progression of the motion based activity.

### EXAMPLE 34

In Example 33 or other examples, the progression of the motion based activity includes a hand path side view or a hand path top view of the motion based activity.

### EXAMPLE 35

In Examples 33, 34 or other examples, the analytics determiner is to perform analytics on the fused first and second motion data to determine performance indicators for the motion based activity.

### EXAMPLE 36

In Example 35 or other examples, the analytics determiner is to determine the performance indicators by identifying velocity peaks within the first motion data and the second motion data to characterize motion of the first joint relative to the second joint during the motion based activity.

### EXAMPLE 37

In Examples 35, 36 or other examples, the display organizer is further to annotate the graphical display to include the performance indicators.

### EXAMPLE 38

In Examples 33, 34, 35, 36, 37 or other examples, the motion data fuser is to fuse the first motion data and the second motion data by applying at least one of an inertial measurement unit algorithm or a fusion algorithm to the first motion data and the second motion data.

### EXAMPLE 39

In Examples 33, 34, 35, 36, 37, 38 or other examples, the motion based activity is a first motion based activity and the progression is a first progression, and further including a comparator is to compare the first progression to a second progression of a second motion based activity.

### EXAMPLE 40

In Example 39 or other examples, the graphical display is a first graphical display, the display organizer is to generate a second graphical display representing the first progression and the second progression.

### EXAMPLE 41

An example method, includes: fusing, by executing an instruction with at least one processor, first motion data and second motion data, the first motion data associated with a first joint on a body and the second motion data associated with a second joint on the body; processing, by executing an instruction with the at least one processor, the fused first and second motion data to identify a progression of a motion based activity; and generating, by executing an instruction with the at least one processor, a graphical display representing the progression of the motion based activity.

### EXAMPLE 42

In Example 41 or other examples, the progression of the motion based activity includes a hand path side view or a hand path top view of the motion based activity.

### EXAMPLE 43

Examples 41, 42 or other examples, the method includes performing, by executing an instruction with the at least one processor, analytics on the fused first and second motion data to determine performance indicators for the motion based activity.

### EXAMPLE 44

In Example 43 or other examples, the performing of the analytics includes identifying velocity peaks within the first motion data and the second motion data to characterize motion of the first joint relative to the second joint during the motion based activity.

### EXAMPLE 45

In Examples 43, 44 or other examples, the method includes annotating, by executing an instruction with the at least one processor, the graphical display to include the performance indicators.

### EXAMPLE 46

In Examples 41, 42, 43, 44, 45 or other examples, the fusing of the first motion data and the second motion data includes applying at least one of an inertial measurement unit algorithm or a fusion algorithm to the first motion data and the second motion data.

### EXAMPLE 47

In Examples 41, 42, 43, 44, 45, 46 or other examples, the motion based activity is a first motion based activity and the progression is a first progression, and further including comparing, by executing an instruction with the at least one processor, the first progression to a second progression of a second motion based activity.

### EXAMPLE 48

In Example 47 or other examples, the graphical display is a first graphical display, further including generating, by executing an instruction with the at least one processor, a second graphical display representing the first progression and the second progression.

### EXAMPLE 49

An example tangible computer-readable medium comprising instructions that, when executed, cause a processor to, at least: fuse first motion data and second motion data, the first motion data associated with a first joint on a body and the second motion data associated with a second joint on the body; process the fused first and second motion data to identify a progression of a motion based activity; and generate a graphical display representing the progression of the motion based activity.

### EXAMPLE 50

In Example 49 or other examples, the progression of the motion based activity includes a hand path side view or a hand path top view of the motion based activity.

### EXAMPLE 51

In Examples 49, 50 or other examples, the instructions, when executed, cause the processor to perform analytics on the fused first and second motion data to determine performance indicators for the motion based activity.

### EXAMPLE 52

In Example 51 or other examples, the performing of the analytics includes identifying velocity peaks within the first motion data and the second motion data to characterize motion of the first joint relative to the second joint during the motion based activity.

### EXAMPLE 53

In Examples 51, 52 or other examples, the instructions, when executed, cause the processor to annotate the graphical display to include the performance indicators.

### EXAMPLE 54

In Examples 49, 50, 51, 52, 53 or other examples, the instructions, when executed, cause the processor to fuse of the first motion data and the second motion data by applying at least one of an inertial measurement unit algorithm or a fusion algorithm to the first motion data and the second motion data.

### EXAMPLE 55

In Examples 49, 50, 51, 52, 53, 54 or other examples, the motion based activity is a first motion based activity and the progression is a first progression, wherein the instructions, when executed, cause the processor to compare the first progression to a second progression of a second motion based activity.

### EXAMPLE 56

In Example 55 or other examples, the graphical display is a first graphical display, wherein the instructions, when executed, cause the processor to generate a second graphical display representing the first progression and the second progression.

### EXAMPLE 57

An example system for use with apparel, comprising: means for fusing first motion data and second motion data, the first motion data associated with a first joint on a body and the second motion data associated with a second joint on the body; means for processing the fused first and second motion data to identify a progression of a motion based activity; and means generating a graphical display representing the progression of the motion based activity.

### EXAMPLE 58

In Example 57 or other examples, the progression of the motion based activity includes a hand path side view or a hand path top view of the motion based activity.

### EXAMPLE 59

In Examples 57, 58 or other examples, the system includes means for performing analytics on the fused first and second motion data to determine performance indicators for the motion based activity.

### EXAMPLE 60

In Example 59 or other examples, the means for performing the analytics includes means for identifying velocity peaks within the first motion data and the second motion data to characterize motion of the first joint relative to the second joint during the motion based activity.

### EXAMPLE 61

In Examples 59, 60 or other examples, the system includes means for annotating the graphical display to include the performance indicators.

### EXAMPLE 62

In Examples 57, 58, 59, 60, 61 or other examples, the motion based activity is a first motion based activity and the progression is a first progression, further including means for comparing the first progression to a second progression of a second motion based activity.

### EXAMPLE 63

In Example 62 or other examples, the graphical display is a first graphical display, further including means for generating a second graphical display representing the first progression and the second progression.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

1. An apparatus for apparel, the apparatus comprising:
a first sensor to be carried at a first location on the apparel to capture first motion data associated with a first part of a body wearing the apparel;
a second sensor to be carried at a second location on the apparel and positioned to capture second motion data associated with a second part of the body; and
a motion monitor to:
compare at least one of the first motion data and the second motion data to reference data to determine when the first and second motion data are associated with a motion based activity; and
cause the first and second motion data to be stored in data storage when the first and second motion data are associated with the motion based activity but not when the first and second motion data are not associated with the motion based activity.

2. The apparatus of claim 1, wherein the first part of the body is a first joint of the body and the second part of the body is a second joint of the body.

3. The apparatus of claim 1, further including a third sensor carried on the apparel at a third location to capture third motion data.

4. The apparatus of claim 3, wherein, the motion monitor is to further compare the third motion data to the reference data to determine when the third motion data is associated with the motion based activity.

5. The apparatus of claim 1, wherein the motion based activity includes hitting a baseball.

6. The apparatus of claim 1, wherein the first sensor and the second sensor are communicatively coupled.

7. The apparatus of claim 1, wherein the first location is one of a wrist area of the smart apparel, a shoulder area of the smart apparel, or a hip area of the smart apparel, and the second location is another one of the wrist area of the smart apparel, a shoulder area of the smart apparel, or a hip area of the smart apparel.

8. The apparatus of claim 1, wherein the motion monitor includes a sensor interface to communicate the first motion data and the second motion data to another device remote from the apparel.

9. The apparatus of claim 1, wherein the first motion data includes first acceleration data reflective of acceleration associated with the first location, first rotation data reflective of rotation associated with the first location, and first position data reflective of a position of the first location during the motion based activity.

10. A method, comprising:
comparing, by executing an instruction with at least one processor, at least one of first motion data and second motion data to reference data to determine when the first and second motion data are associated with a motion based activity, the first motion data associated with a first part of a body wearing apparel, the second motion data associated with a second part of the body wearing the apparel; and
causing, by executing an instruction with the at least one processor, the first and second motion data to be stored in data storage when the first and second motion data are associated with the motion based activity but not when the first and second motion data are not associated with the motion based activity.

11. The method of claim 10, wherein the first part of the body is a first joint of the body and the second part of the body is a second joint of the body.

12. The method of claim 10, further including comparing third motion data to the reference data to determine when the third motion data is associated with the motion based activity, the third motion data associated with a third part of the body wearing the apparel.

13. A tangible computer-readable medium comprising instructions that, when executed, cause a processor to, at least:
compare at least one of first motion data and second motion data to reference data to determine when the first and second motion data are associated with a motion based activity, the first motion data associated with a first part of a body wearing apparel, the second motion data associated with a second part of the body wearing the apparel; and
cause the first and second motion data to be stored in data storage when the first and second motion data are associated with the motion based activity but not when the first and second motion data are not associated with the motion based activity.

14. The computer-readable medium as defined in claim 13, wherein the first part of the body is a first joint of the body and the second part of the body is a second joint of the body.

15. The computer-readable medium as defined in claim 13, wherein the instructions, when executed, cause the processor to compare third motion data to the reference data to determine when the third motion data is associated with the motion based activity, the third motion data associated with a third part of the body wearing the apparel.

16. The computer-readable medium as defined in claim 13, wherein the motion based activity includes hitting a baseball.

17-19. (canceled)

20. An apparatus, comprising:
a data interface to access first motion data and second motion data generated by the smart apparel, the first motion data associated with a first joint on a body and the second motion data associated with a second joint on the body;
a motion data fuser to fuse the first motion data and the second motion data;
an analytics determiner to process the fused first and second motion data to identify a progression of a motion based activity; and
a display organizer to generate a graphical display representing the progression of the motion based activity.

21. The apparatus of claim 20, wherein the progression of the motion based activity includes a hand path side view or a hand path top view of the motion based activity.

22. The apparatus of claim 20, wherein the analytics determiner is to perform analytics on the fused first and second motion data to determine performance indicators for the motion based activity.

23. The apparatus of claim 22, wherein the analytics determiner is to determine the performance indicators by identifying velocity peaks within the first motion data and the second motion data to characterize motion of the first joint relative to the second joint during the motion based activity.

24. The apparatus of claim 22, wherein the display organizer is further to annotate the graphical display to include the performance indicators.

25. The apparatus of claim 20, wherein the motion data fuser is to fuse the first motion data and the second motion data by applying at least one of an inertial measurement unit algorithm or a fusion algorithm to the first motion data and the second motion data.
